(19) United States
(12) Patent Application Publication
(10) Pub. No.: US 2010/0129435 A1
(43) Pub. Date: May 27, 2010

(54) Methods and compositions for treatment of ocular neovascularization and neural injury

(75) Inventors: Larry A. Wheeler, Irvine, CA (US); Gerald W. DeVries, Laguna Hills, CA (US)

Correspondence Address: ALLERGAN, INC., 2525 DUPONT DRIVE, T2-7H, IRVINE, CA, 92612-1599, US

(73) Assignee: Allergan, Inc., Irvine, CA (US)

(21) Appl. No.: 12/381,547

(22) Filed: Mar. 12, 2009

Related U.S. Application Data
(62) Division of application No. 10/020,541, filed on Apr. 26, 2002, now abandoned.
(60) Provisional application No. 60/244,850, filed on Nov. 1, 2000.

(51) Int. Cl. A61K 9/127; A61K 31/13; A61K 38/18; A61K 9/48; A61P 27/02; A61K 31/7088

(52) U.S. Cl. 424/450; 514/662; 514/12; 424/451; 514/44 R

(57) ABSTRACT

Methods and compositions for the treatment of ocular neovascularization and macular degeneration. The invention includes combining photodynamic therapy with administration of a neuroprotectant and a neovascularization inhibitor.

The present application is a divisional of U.S. patent application Ser. No. 10/020,541, filed Oct. 30, 2001 and given an effective filing date of Apr. 26, 2002, which claimed priority to U.S. Provisional application Ser. No. 60/244,850, filed Nov. 1, 2000, the disclosures of each of which are hereby incorporated by reference herein.

### BACKGROUND OF THE INVENTION

Loss of visual acuity is a common problem associated with aging and with various conditions of the eye. Particularly troublesome is the development of unwanted neovascularization in the cornea, retina or choroid. Choroidal neovascularization leads to hemorrhage and fibrosis, with resultant visual loss in a number of recognized eye diseases, including macular degeneration, ocular histoplasmosis syndrome, myopia, diabetic retinopathy and inflammatory diseases.

Age-related macular degeneration (AMD) is the leading cause of new blindness in the elderly, and choroidal neovascularization is responsible for 80% of the severe visual loss in patients with this disease. Although the natural history of the disease is eventual quiescence and regression of the neovascularization process, this usually occurs at the cost of sub-retinal fibrosis and vision loss.

Traditional treatment of AMD relies on occlusion of the blood vessels using laser photocoagulation. However, such treatment requires thermal destruction of the neovascular tissue, and is accompanied by full-thickness retinal damage, as well as damage to medium and large choroidal vessels. Further, the subject is left with an atrophic scar and visual scotoma. Moreover, recurrences are common, and visual prognosis is poor.

Recent research in the treatment of neovascularization have had the aim of causing more selective closure of the blood vessels, in order to preserve the overlying neurosensory retina. One such strategy is a treatment termed photodynamic therapy or PDT, which relies on low intensity light exposure of photosensitized tissues to produce lesions in the newly developing blood vessels. In PDT, photoactive compounds are administered and allowed to reach a particular undesired tissue which is then irradiated with a light absorbed by the photoactive compound. This results in destruction or impairment of the tissue immediately surrounding the locus of the photoactive compound without the more extensive ocular. tissue damage seen when photocoagulation is used.

Photodynamic therapy of conditions in the eye has been attempted over the past several decades using various photoactive compounds, e.g., porphyrin derivatives, such as hematoporphyrin derivative and Photofrin porfimer sodium; “green porphyrins”, such as benzoporphyrin derivative (BPD), MA; and phthalocyanines. Photodynamic treatment of eye conditions has been reported to actually enhance the visual acuity of certain subjects. U.S. Pat. No. 5,756,541.

However, although generally more safe than photocoagulation, there are certain dangers involved in performing PDT. For example, the use of low intesity lasers in conjunction with the systemic injection of vertporfin is currently the only approved PDT for treatment of age-related macular degeneration. But studies have shown that the use of vertporfin at high doses (12 and 18 mg/m³) result in long term or permanent scarring of the retina, chronic absence of photoreceptor cells, and optic nerve atrophy. Reinke et al., Ophthalmology 106:1915 (October 1999), incorporated by reference herein. At lower concentrations of vertporfin (e.g., about 6 mg/m³) PDT is effective to slow vascular outgrowth somewhat, but treatment appears to be necessary every few weeks.

Pigment epithelium-derived factor (PEDF) is a polypeptide originally isolated from cultured fetal human retinal pigment epithelial (RPE) cells. See Tombran-Tink et al., Exp. Eye Res. 53:411-414 (1991), incorporated by reference herein. PEDF and peptide fragments of PEDF have been shown to stimulate the elaboration of neuron-like processes from undifferentiated retinoblastoma cells. The PEDF polypeptide has an approximate molecular weight of 50 kDa. In addition to stimulating morphological changes, PEDF induces differentiation of the retinoblastoma cells. Additionally, PEDF has recently been shown to be an angiogenic inhibitor. Dawson et al., Science 285:245 (9 Jul. 1999), hereby incorporated by reference herein.

In vivo, PEDF is present in the normal mammalian interphotoreceptor matrix (IPM) between the neural retina and the pigment epithelium. The PEDF gene is expressed early (17 weeks of gestation) in human RPE cells, and is thus a prime candidate as an inducer of retinal development in early development. In studies using lung fibroblast cells, the expression of PEDF (also termed EPC-1) has been found be restricted to young cells in the G₀ stage of the cell cycle. In older senescent fibroblast cells PEDF transcripts are absent.

The native PEDF is thought to be a monomeric glycoprotein. The purified native protein is sensitive to glycosidase F, indicating that it contains N-linked oligosaccharides. Upon glycosidase digestion, there is an approximate 3000 Dalton shift in the apparent molecular weight of the protein.

Recombinant forms of PEDF and fragments thereof have been made and expressed in E. coli as well as mammalian cells. The amino acid sequence of human PEDF is provided as SEQ ID NO:1 below:

mqalvlllci gallghsscq npasppeegs pdpdstgalv eeedpffkvp vnklaaaysn fgydlyrvrs smspttnvll splsvatals alslgadert esiihralyy dlisspdihg tykelldtvt apqknlksas rivfekklri kssfvaplek sygtrprvlt gnprldlqei nnwvqaqmkg klarstkeip deisilllgv ahfkgqwvtk fdsrktsled fyldeertvr vpmmsdpkav lrygldsdls ckiaqlpltg smsiifflpl kvtqnitlie esltsefihd idrelktvqa vltvpklkls yegevtkslq emklqslfds pdfskitgkp ikltqvehra gfewnedgag ttpspglqpa hltfpldyhl nqpfifvlrd tdtgallfig kildprgp

This sequence has GenBank accession number AAA60058, the GenBank sequence listing is hereby incorporated by reference herein. In addition, PEDF has been isolated from a variety of other mammalian species, including cattle, mouse and rat; these sequences are also listed in GenBank, and are also incorporated by reference herein.

PE-DF has a sequence homologous to members of the serpin protease inhibitor family. However, PEDF has not been shown to inhibit serine proteases like many serpins. Moreover, the protease labile loop region characteristic of serpins (which is positioned in the carboxyl terminal region of PEDF) is not necessary for the neurotrophic activity of the polypeptide. Experiments have demonstrated that both protease-cleaved native and truncated recombinant forms of PEDF retain neurite differentiating activity, even when the polypeptide (normally 418 amino acids) consists only of as few as the 77 N-terminus proximal residues at positions 44-121. Additionally, incubation of PEDF at 75° C. does not prevent PEDF from differentiating retinoblastoma cells. Becerra et al., J. Biol. Chem. 270:25992 (1995), incorporated by reference herein.

Other neuroprotectant polypeptides have been described. For example, nerve growth factor (NGF) is a polypeptide known to have neuroprotective and neurotrophic effects. Increased survival of photoreceptors in rd mutant mice has been observed upon intravitreal injection of purified NGF; these mice are models of retinitis pigmentosa, a condition characterized by the specific loss of photoreceptors. Lambiase et al., Graefe's Arch. Clin. Exp. Ophthalmol. 234:S96-S100 (1996), hereby incorporated by reference.

Human NGF has an amino acid sequence, from amino to carboxyl terminus, as provided in SEQ ID NO:2 below:

mqaqqyqqqr rkfaaaflaf ifilaavdta eagkkekpek kvkksdcgew qwsvcvptsg dcglgtregt rtgaeckqtm ktqrckipcn wkkqfgaeck yqfqawgecd lntalktrtg slkralhnae cqktvtiskp cgkltkpkpq aeskkkkkeg kkqekmld

NGF sequences are available via the National Center for Biotechnological Information website. This human NGF amino acid sequence is present in the NCBI database under Genbank Accession No. AAA35961.

Also, as disclosed in U.S. Pat. No. 5,958,875, a multimeric cyclic peptide comprising a sequence of amino acid residues or biologically functional equivalents thereof, which are substantially homologous to residues 29-38 of NGF, residues 43-47 of NGF or residues 92-97 of NGF, and further comprising a penicillamine residue or a cysteine residue is also sufficient to have neurotrophic activity. This patent is hereby incorporated by reference herein.

The growth factor ciliary neurotropic factor (CNTF) has been shown to be effective in the protection of photoreceptors in rds/rds mutant mice, another model of retinitis pigmentosa. In one such study, the CNTF was administered via an adenovirus gene transfer vector containing a nucleic acid region comprising an expressible open reading frame encoding the CNTF gene. Cayouette et al., J. Neurosci. 18:9282 (1998), incorporated by reference herein. The adenovirus vector used for these studies was a replication-defective construct lacking the E1 region of the viral genome, and the CFTF gene was fused to the leader sequence of nerve growth factor which directed the protein's secretion from the vector-transduced cells. The vector was administered by intravitreal injection; the amount injected was 2.9×10⁷ plaque forming units (pfu) in 1 ul. The rds/rds mice given this vector displayed greater photoreceptor survival than in animals given a negative control. Additionally, the CNTF expression vector showed greater neuroprotection than in similar animals given an intravitreal injection of recombinant CNTF protein. Thus, the ability of the CNTF expression vector to provide a sustained dosage of CNTF to retinal cells appears to counteract the turnover of the CNTF protein in oculo. The amino acid sequence of human CNTF is provided below as SEQ ID NO:3:
maftehsplt phrrdlcsrs iwlarkirsd ltaltesyvk hqglnkninl dsadgmpvas tdqwseltea erlqenlqay rtfhvllarl ledqqvhftp tegdfhqaih tlllqvaafa yqieelmill eykiprnead gmpinvgdgg lfekklwglk vlqelsqwtv rsihdlrfis shqtgiparg shyiannkkm
   CNTF sequences are available via the National Center for Biotechnological Information website. This human CNTF amino acid sequence is present in the NCBI database under Genbank Accession No. UNHUCF.

Similar results have been described for another nerve cell growth factor, brain derived neurotrophic factor (BDNF). In this case, BDNF cDNA was inserted into a replication-deficient adenovirus vector and injected into the vitreous chamber of adult rats. A subpopulation of retinal glial cells, the Muller cells, expressed and secreted the recombinant BDNF; transgenic protein expression peaked at about 6-7 days following injection of the BDNF expression vector. The eyes treated with the BDNF vectors were effective to rescue injured retinal ganglion cells and these results were superior to a single injection of purified recombinant BDNF. DiPolo et al., Proc. Natl. Acad. Sci. 95:3978 (1998), hereby incorporated by reference herein.

The amino acid sequence of BDNF is given below as SEQ ID NO:4:
mtilfltmvi syfgcmkaap mkeanirgqg glaypgvrth gtlesvngpk agsrgltsla dtfehmieel ldedqkvrpn eennkdadly tsrvmlssqv pleppllfll eeyknyldaa nmsmrvrrhs dparrgelsv cdsisewvta adkktavdms ggtvtvlekv pvskgqlkqy fyetkcnpmg ytkegcrgid krhwnsqcrt tqsyvraltm dskkrigwrf iridtscvct ltikrgr
   BDNF sequences are available via the National Center for Biotechnological Information Website. This human BDNF amino acid sequence is present in the NCBI database under Genbank Accession No. AAA96140.

There are also non-peptide agents known to be neuroprotective. For example, and without limitation, the compounds brimonidine and memantine are neuroprotective agents.

Furthermore, there are neovascularization-inhibiting agents such as, without limitation, the tyrosine kinase inhibitors disclosed in U.S. Pat. No. 6,100,254, hereby incorporated by reference herein, EMD 121974, endostatin, PTK 787, BMS 275291, SU 6668, CGS 27023A, TNP 470, Vitaxin, SU 5416, thalidomide, marimastat, AG 3340, neovasat, anti VEGF antibody, CAI and squalamihe.

### SUMMARY OF THE INVENTION

The present invention concerns compositions and methods for the treatment of ocular neovascularization. In a preferred aspect, the invention is drawn to an improved method of performing photodynamic therapy comprising treating the patient with an effective amount of a neuroprotective agent. Preferably, the neuroprotective agent is selected from the group consisting of nerve growth factor (NGF), ciliary neurotrophic growth factor (CNTF), brain-derived neurotrophic factor (BDNF) and pigment epithelium-derived factor (PEDF). Even more preferably the neuroprotective agent is PEDF.

By “effective amount” of a neuroprotective agent is meant an amount effective to reduce cell death among the neurons of the retina and optic nerve (e.g., photoreceptors) caused by the photoactive component of PDT treatment as compared to a similarly situated PDT patient not receiving treatment with the neuroprotective agent.

In another embodiment, the invention is drawn to an improved method of performing photodynamic therapy comprising treating the patient with an effective amount of a neovascularization-inhibiting agent effective to protect the neurons of the retina and optic nerve (e.g., photoreceptors) from damage caused by the photoactive component of PDT treatment. Preferably, the neovascularization-inhibiting agent is PEDF.

By “effective amount” of a neovascularization-inhibiting agent is meant an amount of such agent effective to reduce the extent to which, or the rate at which, new blood vessels are formed in the retina of a PDT patient as compared to a similarly situated PDT patient not given the neovascularization-inhibiting agent.

In a third embodiment, invention is directed to an improved method of performing photodynamic therapy comprising treating the patient with an effective amount of a neovascularization-inhibiting agent, and with an effective amount of a neuroprotective agent. Preferably, both the neuroprotective agent and the neovascularization-inhibiting agent is PEDF.

In another preferred aspect, the invention is drawn to an improved method of performing photodynamic therapy comprising treating the patient with an amount of PEDF effective to inhibit or block neovascularization so as to increase the amount of time necessary between PDT treatments and to slow the progression of ARMD and other ocular conditions in which neovascularization plays a part beyond that obtained by PDT alone.

When PEDF or another agent having both neuroprotective and antiangiogenic activities is used in conjunction with PDT, it is preferred that the amount of such agent provided to PDT patients is both an effective neuroprotective dose and an effective neovascularization inhibitory dose.

Determining the absolute dosage of the neuroprotective agent and/or neovascularization-inhibiting agent depends upon a number of factors, including the means of administration and delivery and the form of the drug. For intraocular delivery of the purified recombinant PEDF polypeptide, CNTF polypeptide, BDNT polypeptide or NGF polypeptide (or active derivatives and fragments thereof), such as by intravitreal or subretinal injection, dosages are preferably in the range of about 0.1 ug to about 100 ug per eye; more preferably in the range of about 0.20 ug to about 50 ug per eye; even more preferably in the range of about 0.5 ug to about 10 ug per eye.

Whether a neuroprotective agent, a neovascularization-inhibiting agent or both, the agent(s) may be delivered by any means effective to expose the retinal and optic nerve cells to the agent. Thus, such agents may be delivered systemically, such as by intravenous, intramuscular, or subcutaneous injection. Alternatively, the neuroprotective and/or neovascularization-inhibiting agent(s) may be delivered by direct injection into the eye, such as into the anterior chamber, posterior chamber or vitreous chamber, or by subretinal injection.

Another delivery method provides for sustained delivery of the polypeptide using an intraocular implant. Such implants may be, for example, a biodegradable and/or biocompatible implant or insert such as the ocular implants and inserts disclosed in U.S. Pat. Nos. 5,443,505, 5,824,072, 5,766,242; 4,853,224; 4,997,652; 5,164,188; 5,632,984; and 5,869,079, incorporated by reference herein. Such implants may be inserted into a chamber of the eye, such as the anterior, posterior or anterior chambers, or may be implanted in the schlera, transchoroidal space, or an avascularized region exterior to the vitreous.

Other methods for the delivery of polypeptide neuroprotective and/or antiangiogenic agents, such as PEDF, BDNF, CNTF, or NGF include a gene therapy vector, such as an adenovirus vector, which comprises a therapeutic nucleic acid comprising an open reading frame encoding the thereapeutic agent (or an active fragment thereof) which is capable of being expressed in a target cell, such as retinal endothelium cells. Such vectors have been made and have been widely employed in basic research and in clinical trials of therapeutic proteins. In an aspect of the present invention, the delivery of the proteinacious neuroprotective and/or antiangiogenic agent(s)(or therapeutic nucleic acids encoding active fragments of such a polypeptide agent, such as the PEDF, BDNF, NGF, CNTF or BDNF proteins or derivatives or active fragments thereof) is facilitated by delivering the vector directly to the vitreous of the eye, e.g., by injection using a narrow gauge hypodermic needle or capillary tube. This mode of treatment has the advantage of delivering the therapeutic agent precisely to the desired retinal site of action, while reducing the necessary total dose as compared to systemic delivery of the viral vector. Adenoviral vectors are usually capable of transient expression over a period of days or weeks. Such time periods are consistent with use in conjunction with PDT. The amount of the vector delivered may be in the order of about 3.0×10⁷ pfu in a volume of about 0.5-5 ul. If the initial dose is not a consideration, then a PEDF-containing expression vector may alternatively be delivered systemically, for example by intravenous infusion or intramuscular or subcutaneous injection.

### DETAILED DESCRIPTION OF THE INVENTION

The present invention is drawn to therapeutic methods and compositions for the treatment of intraocular neovascularization associated with conditions such as age-related macular degeneration (ARMD) and diabetic retinopathy.

The invention is more particularly concerned with therapeutic methods combining retinal photodynamic therapy (PDT) with a neuroprotectant agent and/or an inhibitor of neovascularization; preferably with a single agent having both of these activities. In a preferred embodiment, the agent is a single agent having PEDF activities. In a currently more preferred aspect, the agent is human PEDF.

In a preferred aspect of this embodiment of the invention, the neuroprotective and/or antiangiogenic agent(s) are administered to the patient sufficiently prior to PDT treatment so as to be available to protect nerve cells and/or inhibit neovascularization upon the commencement of therapy. In another aspect of the invention, PEDF is administered with sufficient time to inhibit or block neovascularization occurring after PDT treatment.

Such methods are applicable to PDT treatment which makes use of any photoactive compound. Such compounds may include derivatives of hematoporphyrin, as described in U.S. Pat. Nos. 5,028,621; 4,866,168; 4,649,151; and 5,438,071. pheophorbides are described in U.S. Pat. Nos. 5,198,460; 5,002,962; and 5,093,349; bacteriochlorins in U.S. Pat. Nos. 5,171,741 and 5,173,504; dimers and trimers of hematoporphyrins in U.S. Pat. Nos, 4,968,715 and 5,190,966. Other possible photoactive compounds include purpurins, merocyanines and porphycenes. All of the aforementioned patents are incorporated by reference herein. Of course, mixtures of photoactive compounds may be used in conjunction with each other.

A currently preferred photoactive compound is verteporfin (liposomal benzoporphyrin derivative). This compound is currently the only photoactive agent approved by the U.S. Food and Drug Administration for treatment of choroidal neovascularization in conjunction with photodynamic therapy.

The photoactive agent is formulated so as to provide an effective concentration to the target ocular tissue. The photoactive agent may be coupled to a specific binding ligand which may bind to a specific surface component of the target ocular tissue, such as a cell surface receptor or, if desired, may be formulated with a carrier that delivers higher concentrations of the photoactive agent to the target tissue. Exemplary ligands may be receptor antagonists or a variable region of an immunoglobulin molecule.

The nature of the formulation will depend in part on the mode of administration and on the nature of the photoactive agent selected. Any pharmaceutically acceptable excipient, or combination thereof, appropriate to and compatible with the particular photoactive compound may be used. Thus, the photoactive compound may be administered as an aqueous composition, as a transmucosal or transdermal composition, or in an oral formulation. The formulation may also include liposomes. Liposomal compositions are particularly preferred especially where the photoactive agent is a green porphyrin. Liposomal formulations are believed to deliver the green porphyrin with a measure of selectivity to the low-density lipoprotein component of plasma which, in turn acts as a carrier to deliver the active ingredient more effectively to the desired site. Increased numbers of LDL receptors have been shown to be associated with neovascularization, and by increasing the partitioning of the green porphyrin into the lipoprotein phase of the blood, it appears to be delivered more efficiently to neovasculature.

Consistent with the chosen formulation, the photoactive compound may be delivered in a variety of ways. For example, delivery may be oral, peritoneal, rectal, or topical (e.g., by installation directly into the eye). Alternatively, delivery may be by intravenous, intramuscular or subcutaneous injection.

The dosage of the photoactive compound may vary, according to the activity of the specific compound(s) chosen, the formulation, and whether the compound is joined to a carrier and thus targeted to a specific tissue as described above. When using green porphyrins, dosages are usually in the range of 0.1-50 mg/M² of body surface area; more preferably from about 1-10 mg/M² or from about 2-8 mg/M². Obviously, parameters to be considered when determining the dosage include the duration and wavelength of the light irradiation and the nature of the photochemical reaction induced by the light irradiation.

Light irradiation is performed a sufficient time after the administration of the photoactive compound so as to permit the compound to reach its target tissue. Upon being irradiated with the wavelength appropriate to the compound chosen, the compound enters an excited state and is thought to interact with other compounds to form highly reactive intermediates which can then destroy the target endothelial tissue, causing platelet aggregation and thrombosis. Fluence of the irradiation may vary depending on factors such as the depth of tissue to be treated and the tissue type—generally it is between about 50 and about 200 Joules/cm². Irradiance typically is between about 150 and about 900 mW/cm², but can also vary somewhat from this range.

Typically, light treatment is given about two hours following administration of the photoactive drug. In a preferred embodiment, the photoactive drug is administered intravenously.

The other component(s) of the methods and composition of the present invention are a neuroprotective and/or a neovascularization-inhibiting agent. Exemplary neuroprotective agents are, without exception, NGF, BDNF, CNTF and PEDF. Exemplary neovascularization-inhibiting agents are, without limitation, PEDF. NGF, BDNF, CNTF and PEDF have all been shown to have strong neurotrophic activity.

In a preferred aspect of the invention both a neuroprotective and neovascularization-inhibiting agent are administered to the eye to protect it during and after PDT treatment. In an even more preferred embodiment of the invention, the neuroprotective and neovascularization-inhibiting agent is a single compound. In a most preferred embodiment of the invention, the single compound is PEDF.

PEDF prolongs the life of brain neurons in culture and protects neurons against acute neurotoxic insult due to, e.g., glutamate toxicity. Thus, PEDF appear to protect neurons against programmed cell death. PEDF is also an inhibitor of neovascularization. Further, PEDF appears to promote the differentiation of immature of neural lineage into neurons and studies have shown that it is capable of deterring the onset of cellular senescence.

The neuroprotective and/or neovascularization-inhibiting agent(s) of the present invention are delivered in any manner in which it is effective to protect neurons and/or inhibit neovascularization incident to PDT treatment. Generally, the agent(s) is administered prior to PDT treatment, so as to permit it to reach the ocular neural tissue before phototherapy. This will permit the agent(s) to have an immediate protective effect on neural cells. However, the neovascularization-inhibiting benefits of an antiangiogenic agent such as PEDF can be realized even when given simultaneously with, or shortly after PDT treatment.

It will be recognized that the term PEDF means biologically active PEDF and its biologically active derivatives, particularly peptides containing a region of contiguous amino acids within the region corresponding to positions 44-267, preferably within the region 44-229, most preferably within the region 44-121 of the human PEDF polypeptide. Preferably the PEDF has an amino acid sequence contained in the human PEDF amino acid sequence.

Purified native, wild-type PEDF may be used as the therapeutic agent in the methods and compositions of the present invention. Bovine PEDF has been purified to apparent homogeneity from the vitreous body of eyes, using an ammonium sulfate precipitation step (45% to 80%), followed by cation exchange chromatography (e.g., Mono-S chromatography in a 100 mM to 500 mM salt gradient). A similar purification protocol may be effective to purify the polypeptide from human fetal retinal pigment epithelium cell culture conditioned medium.

Alternatively, the PEDF cDNA may be cloned and expressed in mammalian, insect, or bacterial cells. Recombinant human full length PEDF, and truncated forms thereof have been expressed in E. coli; the recombinant proteins retain biological activity in vitro despite presumably having different or absent glycosylation from native PEDF. Purification from bacterial cells can be facilitated by permitting the PEDF polypeptides to accumulate at high yield in inclusion bodies, which can then be isolated, solubilized in 4 M urea, and purified by S-Sepharose chromatography in a linear NaCl gradient.

As indicated above, PEDF may be formulated in any manner effective to stabilize the polypeptide and consistent with the delivery method. Since the PEDF polypeptide has been shown to retain its biological activity upon incubation at 75° C., the core neurotropically-active PEDF protein is hardy and tolerant to formulation using methods that might tend to denature other proteins.

Additionally, PEDF may be joined, in a manner similar to that of the photoactive compounds, to cell surface targeting ligands, such as portions of an antibody or immunologically active fragments to aid in targeting the polypeptide to ocular cells, such as the optic nerve neurons and photoreceptors.

PEDF may be formulated for oral delivery in, for example, a capsule, tablet or liquid. Particularly when formulated in solid form, the shelf life of the PEDF may be extended by, for example, lyophilization in an appropriate cryoprotectant. Preferred cryoprotectants are, for example, non-reducing disaccharides. A particularly preferred cryoprotectant is the sugar trehalose.

PEDF may formulated for intravenous, intramuscular, or subcutaneous injection. In such a formulation, any suitable excipient may be added to such a formulation to stabilize the active ingredient and, particularly in the case of intravenous administration, to provide the necessary electrolyte balance.

PEDF may also be formulated as a suppository or otherwise administered rectally. Formulations appropriate for rectal drug administration are well-known to those of skill in the art.

In yet another embodiment PEDF may delivered as a nucleic acid encoding PEDF, which is then transcribed within the target ocular cells. This approach has the advantage that a single nucleic acid may give rise to many molecules of PEDF. The PEDF-encoding nucleic acid may be formulated within liposomes. The liposomes are then able to fuse with a cell membrane, thus delivering the nucleic acid within the cell.

A possibly more efficient means of administering a nucleic acid encoding PEDF is through use of a viral vector. In such a vector, the PEDF-encoding nucleic acid is expressed within the target cell and thereby the PEDF is synthesized and performs its therapeutic action in situ. Moreover, since the PEDF nucleic acids are delivered in a virus “package” the therapeutic nucleic acid is rendered relatively resistant to degradation by the patient's immune system or any nucleases that may be present in the blood or lymph.

Essentially, such delivery methods first involve the choice of an appropriate virus vector. There are a number of considerations in such a choice. For example, the chosen virus must be able to infect the appropriate cell type (e.g., preferably retinal epithelial cells, which can then secret the PEDF thus produced).

Additionally, the vector itself should have low intrinsic toxicity. This term encompasses pharmacological toxicity, immune responses to the vector, the passenger gene product, or any other genes expressed by the vector in situ.

Studies have been performed using modified vectors derived from viruses such as adenovirus and adeno-associated virus (AAV-2). Of course, other applicable viral vectors are available or can be envisioned by the person of ordinary skill in the art; the vectors mentioned herein are by way of illustration rather than limitation.

Each prospective vector has its own properties. For example, adenovirus infections are common and relatively benign in humans; this virus is one of those responsible for the common cold. The virus contains a double-stranded DNA genome. After deletion of non-essential genes, the virus is able to carry about 8 kilobase pairs of an exogenous double-stranded DNA insert. This amount is adequate to carry PEDF coding regions and any necessary regulatory sequences, such as those responsible of the expression, processing, or secretion of the therapeutic gene product. Such regulatory sequences are well known by those of ordinary skill in the art. Adenovirus does not stably integrate into the host chromosome, and therefore expression of the PEDF gene is relatively transient. Expression of the therapeutic protein in adenovirus systems can be seen soon after infection. Certain constructs of adenovirus (and other gene transfer vectors) have been made “replication deficient” in order to control the extent and duration of infection.

AAV-2 also commonly infects humans but is not known to cause a disease. The virus is quite small, and therefore it is relatively non-immunogenic.

However, the small size also means that there is less room for packaging therapeutic genes and any necessary regulatory sequences. Wild-type AAV-2 stably integrates at a specific site in human chromosome 19, however the gene responsible for stable integration is deleted in recombinant versions of the viral genome, and this property is therefore lost.

PEDF, as indicated above, and nucleic acids encoding PEDF and variants may be administered by systemic delivery, as by intravenous, intramuscular or subcutaneous injection. In addition, these factors may be delivered directly to the eye by biocompatable and/or biodegradable implants or inserts (such as those described in patents cited and incorporated by reference above) containing the protein or nucleic acid, or by direct injection into the eye, for example by intravitreal and/or subretinal injection. Alternatively, the PEDF may be topically applied to the surface in an drop.

The therapeutically effective PEDF dosage will depend upon factors including the mode of delivery, the specific activity of the polypeptide, the formulation in which the PEDF is fabricated, and the form of PEDF, whether the full length polypeptide or truncated forms thereof or a nucleic acid form. Once a formulation and route of administration is decided upon, determining a therapeutically effective dose is routine in the pharmaceutical arts, and can be readily determined without undue experimentation using suitable animal models such as, without limitation, non-human primates and rabbits.

Preferably, the dosage regimen of either or both the neuroprotective and antiangiogenic agent will be such to permit the active agent(s) to remain in contact with retinal cells throughout the treatment period. Thus, the agent may be administered, for example, once a week for 12 weeks. If an agent is a polypeptide, like PEDF, susceptible to proteolytic cleavage, the agent may be administered more frequently. An advantage of providing the agent in the form of an expressible gene is that the frequesncy of administration can be reduced, as the active agent is constantly produced so long as the vector is capable of expression.

Viral vectors are constructed using standard molecular biological techniques employed by those of skill in the art. For example, U.S. Pat. Nos. 6,083,750 and 6,077,663 are drawn to improved adenovirus-based expression vectors. These patents, including their descriptions of preparing viral vectors for heterologous gene expression in mammalian cells, are hereby incorporated herein by reference.

### Example 1

A 74 year old patient presents with “wet” age-related macular degeneration (ARMD) in the foveal region of the right eye, and his condition is found to be suitable for photodynamic therapy (PDT). One day prior to the date of scheduled treatment, the patient is given an intravenous injection of PEDF in a standard infusion solution.

The day of scheduled PDT treatment, the patient is administered 6 mg/M² of verteporfin. Thirty minutes after the start of the infusion, the patient is administered Irradiance of 600 mW/cm² and total fluence of 75 Joules/cm² from an Argon light laser. The treatment requires irradiation of the optic nerve.

PEDF administration is continued every two days throughout the 12 week evaluation period.

Evaluation of neural health is assayed 1 week, 4 weeks, and 12 weeks following treatment by visual inspection of the retina and test of visual acuity. The affected areas of the retina appear healthy with no whitening (indicating lack of discernable retina damage) one week following PDT treatment; this trend continues throughout the monitoring period. Fluorescein angiography at same time points shows minimal leakage in the treated tissue after one week, and this minimal leakage continues throughout the monitoring period. No evidence of renewed neovascularization can be seen 12 weeks following PDT treatment. Additionally, no evidence of optic nerve axon loss can been seen. Tests of visual acuity 4 and 12 weeks following combined PDT and PEDF treatment show no discernable loss of vision as a result of the treatment.

### Example 2

Same facts as in Example 1, except that rather than being given intravenous PEDF, the patient is given a replication-deficient adenovirus vector containing an expressible PEDF gene containing the signal sequence for NGF. The vector is administered by intravitreal injection three days prior to PDT treatment (3×107 pfu per eye in 1 ul); the vector is readministered 5 days following PDT treatment and every week thereafter for the 12 week evaluation period.

Evaluation of neural health is assayed 1 week, 4 weeks, and 12 weeks following treatment by visual inspection of the retina and test of visual acuity. The affected areas of the retina appear healthy with no whitening (indicating lack of discernable retina damage) one week following PDT treatment; this trend continues throughout the monitoring period. Fluorescein angiography at same time points shows minimal leakage in the treated tissue after one week, and this minimal leakage continues throughout the monitoring period. No evidence of renewed neovascularization can be seen 12 weeks following PDT treatment. Additionally, no evidence of optic nerve axon loss can been seen. Tests of visual acuity 4 and 12 weeks following combined PDT and PEDF treatment show no discernable loss of vision as a result of the treatment.

The example illustrates certain embodiments of the present invention; however, it will be understood that the invention is solely defined by the claims that conclude this specification.

We claim:

1) A method of protecting ocular neural tissue from damage caused by photoreaction of a photoactive compound comprising delivering to a patient's ocular neural tissue an amount of a neuroprotectant compound selected from the group consisting of NGF, PEDF, CNTF, BDNF, and memantine effective to protect a plurality of ocular neurons from cell death due to the photoreaction of said photoactive compound as compared to ocular neuron cell death observed under otherwise identical conditions in the absence of the administration of said neuroprotectant.

2) The method of claim 16 wherein said neuroprotectant compound is administered at a time sufficiently before exposure to said photoactive compound to permit localization within ocular tissue prior to said treatment.

3) The method of claim 16 wherein said neuroprotectant compound is administered intravenously.

4) The method of claim 16 wherein said neuroprotectant compound is administered through intraocular injection.

5) The method of claim 14 wherein said neuroprotectant compound is administered by subretinal injection.

6) The method of claim 14 wherein said neuroprotectant compound is administered by intravitreal injection.

7) The method of any one of claim 17 wherein said neuroprotectant compound comprises a recombinant human polypeptide.

8) The method of claim 23 wherein said neuroprotectant compound comprises a continuous amino acid sequence corresponding to positions 44-121 of native human PEDF.

9) The method of claim 24 wherein said PEDF comprises a continuous amino acid sequence corresponding to positions 44-229 of native human PEDF.

10) The method of claim 25 wherein said PEDF comprises a continuous amino acid sequence corresponding to positions 44-267 of native human PEDF.

11) The method of claim 1 wherein said neuroprotective agent is a polypeptide and is administered in the form of a composition comprising a nucleic acid which comprises an open reading frame encoding said neuroprotective agent, and wherein said agent is expressed in ocular tissue.

12) The method of claim 11 wherein said composition comprises a viral coat encapsulating said nucleic acid.

13) The method of claim 11 wherein said composition comprises a liposomal formulation.

14) The method of claim 1 also comprising administering a therapeutically effective amount of a antiangiogenic compound.

15) The method of claim 14 wherein said neuroprotective compound and said antiangiogenic compound are the same compound.

16) The method of claim 15 wherein said compound is PEDF.

17) The method of claim 1 wherein said neuroprotective compound is memantine.


SEQUENCE LISTING (4 sequences)

SEQ ID NO: 1 - protein, 418 aa, Artificial Sequence [sequence omitted]

SEQ ID NO: 2 - protein, 168 aa, Artificial Sequence
  Homo sapiens
MQAQQYQQQRRKFAAAFLAFIFILAAVDTAEAGKKEKPEKKVKKSDCGEWQWSVCVPTSGDCGLGTREGTRTGAECKQTMKTQRCKIPCNWKKQFGAECKYQFQAWGECDLNTALKTRTGSLKRALHNAECQKTVTISKPCGKLTKPKPQAESKKKKKEGKKQEKMLD

SEQ ID NO: 3 - protein, 200 aa, Artificial Sequence
  Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 4 - protein, 247 aa, Artificial Sequence [sequence omitted]